(19) United States
(12) Patent Application Publication
(10) Pub. No.: US 2011/0021950 A1
(43) Pub. Date: Jan. 27, 2011

(54) CELL COLLECTOR

(75) Inventor: Robert L. Daniels, Jamestown, NY (US)

Correspondence Address: Stephen B. Salai, Esq.;Harter Secrest & Emery LLP, 1600 Bausch & Lomb Place, Rochester, NY, 14604-2711, US

(73) Assignee: Father Judge Apostolic Land Company, LLC, Jamestown, NY (US)

(21) Appl. No.: 12/507,339

(22) Filed: Jul. 22, 2009

(51) Int. Cl. A61B 10/02

(52) U.S. Cl. 600/569; 600/562

(57) ABSTRACT

A medical device includes a sheath defining at least one lumen and a probe movably disposed within the lumen. The probe includes a removable handle at a proximal end thereof, a collector at a distal end thereof, and a stop disposed between the proximal end and the distal end. The medical device also includes a preservative disposed within the lumen.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A “SEQUENCE LISTING”

Not applicable.

### BACKGROUND OF THE INVENTION

1. Field of the Invention

The present disclosure generally relates to medical devices and, in particular, to devices useful in collecting and storing target cells or tissues.

2. Description of Related Art

Medical testing procedures are a common, well-known aspect of maintaining ones personal health and well being. Since it can be useful to identify and/or treat illnesses or diseases at an early stage, medical testing procedures are often designed as precautionary measures to be performed on a regular basis.

Despite the known advantages of identifying illnesses and diseases at an early stage, however, many people do not undergo regular medical examinations performed by medical personnel (i.e., physicians, nurses, etc.). In fact, even after notable physical symptoms arise, many people still do not or cannot have an appropriate medical examination performed. Reasons for this vary. For example, some medical testing procedures may be embarrassing, uncomfortable, time consuming, and/or expensive. For example, some people may not have medical insurance, or the medical insurance in place may not cover the required medical testing procedure. Furthermore, some people may live in relatively remote locations, thereby making it more difficult to visit a medical professional to seek care.

Known statistics show that a great many people die each year due to their failure to have medical testing regularly performed for reasons such as those mentioned above. To address this tragic fact, engineers, physicians, and other experts have developed medical testing procedures and devices over the years making it possible for people to perform self-evaluations. For example, known devices exist whereby women can perform medical procedures such as pap smears in the comfort of their own homes. However, such known devices are often cumbersome to handle and transport after such a medical test has been performed. In addition, known testing devices do not provide a reliable means for transporting cell tissue or other material obtained during the testing procedure for evaluation by a medical professional. For example, although known devices may enable women to remove and/or otherwise obtain cervical tissue during a self-administered pap smear, such devices are not easily mailed or otherwise transported to a hospital or a physician's office for evaluation of the cells. Moreover, cervical cells obtained by such devices are not easily preserved within such devices during transport. In fact, such devices typically do not provide a safe, sterile environment for such cells once the cells have been removed from the woman's body. As a result, the cells can become contaminated, particularly if the medical device must be mailed or otherwise shipped to the medical professional for evaluation.

The devices and methods disclosed herein are directed towards overcoming at least some of the above drawbacks associated with the prior art.

### BRIEF SUMMARY OF THE INVENTION

In an exemplary embodiment of the present disclosure, a medical device includes a sheath defining at least one lumen and a probe moveably disposed within the lumen. The probe includes a removable handle at a proximal end thereof, a collector at a distal end thereof, and a stop disposed between the proximal end and the distal end. The medical device also includes a preservative disposed within the lumen.

In an additional exemplary embodiment of the present disclosure, a medical device includes a sheath defining a lumen and a probe disposed within the lumen. The probe includes a collector at a distal end thereof, a break-away handle at a proximal end thereof, and a stop disposed between the collector and the handle. The medical device also includes a preservative substantially filling an inner diameter of the lumen around the probe.

In a further exemplary embodiment of the present disclosure, a method of collecting a target cell from an anatomical structure includes inserting a medical device into a body canal. In such an exemplary embodiment, the medical device includes a sheath defining at least one lumen and a probe disposed within the lumen. The probe includes a removable handle at a proximal end thereof, a collector at a distal end thereof, and a stop disposed between the proximal end and the distal end. The medical device also includes a preservative within the lumen. In such an exemplary embodiment, the method includes collecting the target cell with the collector, disposing the target cell within the preservative, and removing the handle from the probe.

### BRIEF DESCRIPTION OF THE SEVERAL VIEWS OF THE DRAWINGS

FIG. 1 is a partial cross-sectional view of a medical device according to an exemplary embodiment of the present disclosure.

FIG. 2 is another cross-sectional view of the medical device illustrated in FIG. 1.

FIG. 3 is a further cross-sectional view of the medical device illustrated in FIG. 1.

FIG. 4 is a side view of the medical device of FIG. 1 at least partially inserted into a human orifice, according to an exemplary embodiment of the present disclosure.

### DETAILED DESCRIPTION OF THE INVENTION

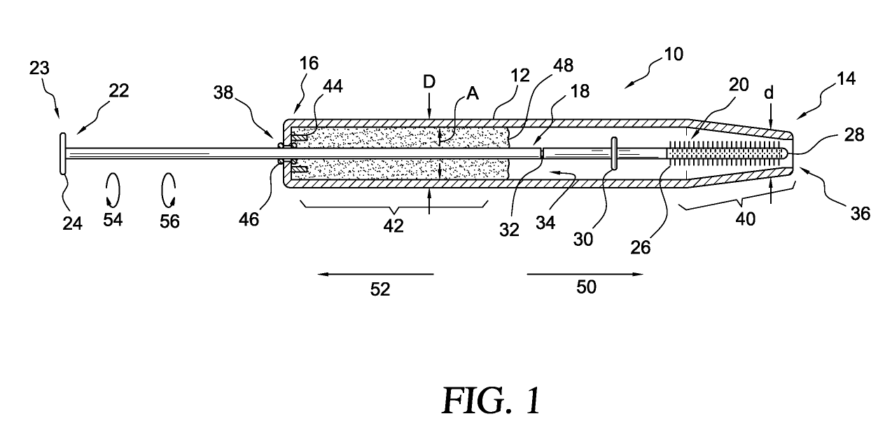

As shown in FIG. 1, a medical device 10 according to an exemplary embodiment of the present disclosure may include, among other things, a sheath 12 defining at least one lumen 34, a probe 18 slideably and/or otherwise moveably disposed within the lumen 34, and a preservative 48 disposed within the lumen 34. The medical device 10 may be, for example, a self-pap smear device, a self-sexual transmitted disease test device, or any other form of medical testing or evaluation device known in the art. Although not specifically mentioned herein, other uses of the medical device 10, such as, for example, capturing, removing, and/or otherwise obtaining target cells through an anatomical orifice are within the scope of the present disclosure.

The sheath 12 may be, for example, substantially cylindrical, and may be made from any hypoallergenic, atraumatic, and/or non-irritave material known in the art. For example, the sheath 12 may be made from metal, plastic, rubber, polymers, alloys, or other known materials. The sheath 12 may have any size, shape, rigidity, and/or other configuration useful for guiding insertion of the medical device 10 and/or the probe 18 into an anatomical orifice or opening such as, for example, a vagina. In an exemplary embodiment, at least a portion of the sheath 12 may be tapered to assist in inserting the medical device 10 into such an orifice. For example, the sheath 12 may include a distal end 14 and a proximal end 16, and a portion 40 of the distal end 14 may have an outer diameter d less than an outer diameter D of a length 42 of the sheath 12. It is understood that the length 42 may be disposed anywhere along the sheath 12 and, in an exemplary embodiment, the length 42 may be proximate the proximal end 16 of the sheath 12.

The sheath 12 may define a distal opening 36 and a proximal opening 38. In an exemplary embodiment, both the distal opening 36 and the proximal opening 38 may be fluidly connected to the lumen 34. In an additional exemplary embodiment in which the sheath 12 defines more than one lumen 34, it is understood that the sheath 12 may also define more than one corresponding distal or proximal openings fluidly connected to such lumens. The distal opening 36 may be sized, shaped, and/or otherwise configured to permit at least a portion of the probe 18 to extend beyond the distal end 14 of the sheath 12 during use. For example, during use, the probe 18 may be moved in the direction of arrow 50 to extend from the distal end 14, or in the direction of arrow 52 to be retracted within the lumen 34. It is understood that the handle 22 may also be rotated in, for example, the direction of arrows 54, 56 as desired, and the distal and proximal openings 36, 38 may be shaped, sized, and/or otherwise configured to facilitate manipulation and/or movement of the probe 18 relative to the sheath 12.

The sheath 12 may be configured to form a substantially fluid-tight seal with, for example, at least a portion of the probe 18. Such a seal may substantially prohibit, for example, preservative 48 from exiting the proximal opening 38 of the sheath 12. In an additional exemplary embodiment, such a substantially fluid-tight seal between the probe 18 and the sheath 12 may also be formed at the distal end 14 of the sheath 12. It is understood that the proximal end 16 and/or the distal end 14 of the sheath 12 may include any of a plurality of components forming such a substantially fluid-tight seal. Although shown in FIG. 1 as including a gasket 46 at the proximal opening 38, it is understood that, in additional exemplary embodiments, such components may include, for example, o-rings, wipers, fins, ribs, and/or other known sealing components. As shown in FIG. 1, at least a portion of the gasket 46 may be disposed within the lumen 34 of the sheath 12 while another portion of the gasket 46 may be disposed proximate an outer surface of the sheath 12. Alternatively, portions of the gasket 46 may be removed. In still another exemplary embodiment, other and/or additional sealing components may be added to the medical device 10 to form the substantially fluid-tight seal discussed above.

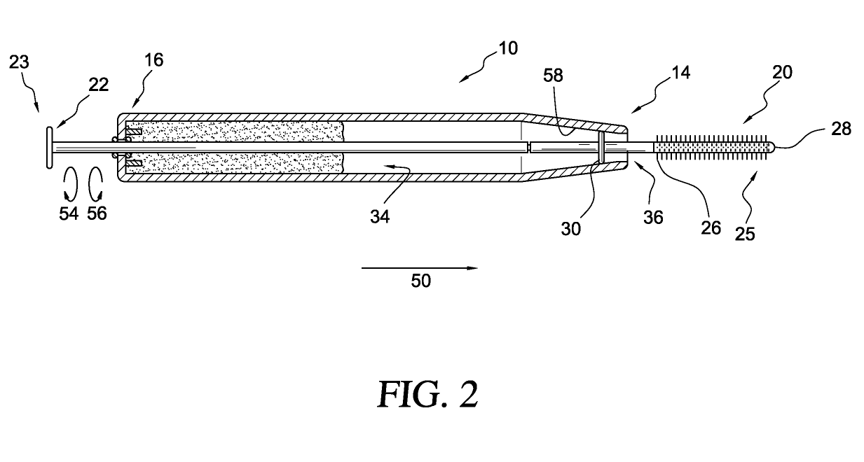

The sheath 12 may also define one or more bumpers 44 disposed within the lumen 34 proximate the proximal end 16 of the sheath 12. The bumpers 44 may be made from any of the materials discussed above with respect to the sheath 12. It is understood that the bumpers 44 may be molded with, adhered to, etched from, and/or otherwise connected to the sheath 12 at the proximal end 16. The bumpers 44 may be sized, shaped, and/or otherwise configured to mate with and/or otherwise abut a portion of the probe 18. For example, as will be discussed in greater detail below, the bumpers 44 may be configured to mate with a stop 30 of the probe 18 when the probe 18 is in a storage position. The bumpers 44 may limit the movement of the probe 18 in the direction of arrow 52 and may maintain at least a portion of the distal end 25 (FIG. 2) of the probe 18 within the lumen 34.

With continued reference to FIG. 1, at least a portion of the probe 18 is disposed within the lumen 34. As noted above, the probe 18 includes a proximal end 23 and a distal end 25 (FIG. 2). The probe 18 may also include a stop 30 disposed between the proximal end 23 and the distal end 25. The probe 18 may further include a handle 22 disposed at the proximal end 23, a weakened section 32 disposed proximal the stop 30, and a collector 20 at the distal end 25.

The collector 20 may be, for example, any structure or other component known in the art configured to assist in collecting, scraping, sampling, and/or removing, for example, at least one target cell from an anatomical location. Such collectors 20 may be useful in, for example, collecting and/or removing one or more cells from a female cervix. A collector 20 may include an agitator 28 disposed at the distal end 25 to assist in collecting and/or removing such target cells. The agitator 28 may be made from, for example, any of the materials discussed above with respect to the sheath 12 and/or the probe 18. For example, the probe 18 and/or the agitator 28 may be made from hypoallergenic and/or atraumatic materials such as, for example, metals or alloys thereof, plastics or polymers, and/or rubber. The agitator 28 may include, for example, a knot, loop, cup, point, and/or any other shape or component disposed at a tip thereof to assist in the collection and/or removal of such target cells. For example, in an exemplary embodiment in which the probe 18 is made from at least one strand of metal wire, the agitator 28 may include a knot formed at a distal tip of the agitator 28 to facilitate the collection and/or removal of target cells.

The collector 20 may further comprise a plurality of bristles 26. The bristles 26 may be made from any of the materials discussed above with respect the sheath 12 and/or the probe 18. The bristles 26 may be substantially atraumatic such that contacting portions of the anatomical structures with the bristles 26 shall not be harmful to the anatomical structure and shall not cause substantial damage thereto while, at the same time, providing for the safe and relatively pain-free removal of target cells therefrom. Such bristles 26 are well known in the art and, accordingly, their construction shall not be described in greater detail herein. The bristles 26 may be disposed in any desirable pattern along the collector 20 to assist in collecting and/or removing such target cells, and such exemplary patterns may be, for example, linear, spiral-shaped, and/or random. In an exemplary embodiment, the collector 20 may be a cytobrush. Alternatively, the collector 20 may resemble the tip of a mascara brush utilized for applying mascara or other beautification products. The collector 20 may be rigidly connected to the probe 18 at the distal end 25. Alternatively, the collector 20 may be formed from the probe 18 such that the distal end 25 of the probe 18 may have a substantially one-piece construction.

The stop 30 may be disposed proximal the collector 20, and may be formed from any of the materials discussed above with respect to the probe 18. For example, the stop 30 may be made from metal or alloys, plastic or polymers, and/or rubber. The stop 30 may be formed from the probe 18 itself and may, thus, have a one-piece construction with the probe 18. Alternatively, the stop 30 may soldered, molded, fused, adhered, and/or otherwise connected to the probe 18 as desired. The stop 30 may have any shape, size, and/or other configuration to enable free movement of the probe 18 within the lumen 34. The stop 30 may also be configured to prohibit removal of the probe 18 from the lumen 34 in the direction of arrow 52. In an exemplary embodiment, the stop 30 may be configured to abut and/or otherwise contact one or more of the bumpers 44 discussed above, thereby providing a mechanical stop to retard movement of the probe 18. Alternatively, in an exemplary embodiment in which the one or more bumpers 44 have been omitted, the stop 30 may be configured to abut, for example, the proximal end 16 of the sheath 12 to provide such a mechanical stop to movement of the probe 18.

The stop 30 may be substantially disk shaped, substantially linear, and/or any other configuration to enable movement of the probe 18 and to minimize the amount of preservative 48 displaced as the probe 18 is moved in, for example, the direction of arrow 52. In an exemplary embodiment, the stop 30 may define one or more holes, perforations, notches, grooves and/or orifices to facilitate, for example, passage of the preservative 48 substantially through portions of the stop 30. In still a further exemplary embodiment, the stop 30 may comprise a rod, cross, or mesh-like structure to assist in minimizing displacement of the preservative 48. In such an exemplary embodiment, the stop 30 may further include a retainer ring (not shown) or other like structure to maintain a desired level of rigidity for abutment with portions of the sheath 12.

In addition, as shown in FIG. 2, the stop 30 may also be configured to retard and/or restrict movement of the probe 18 in the direction of arrow 50. In such an exemplary embodiment, the stop 30 may abut, for example, an inner wall 58 of the sheath 12 proximate the distal end 14. Alternatively, the sheath 12 may further include one or more additional bumpers 44 (not shown) at the distal end 14 configured to abut the stop 30 upon contact therewith.

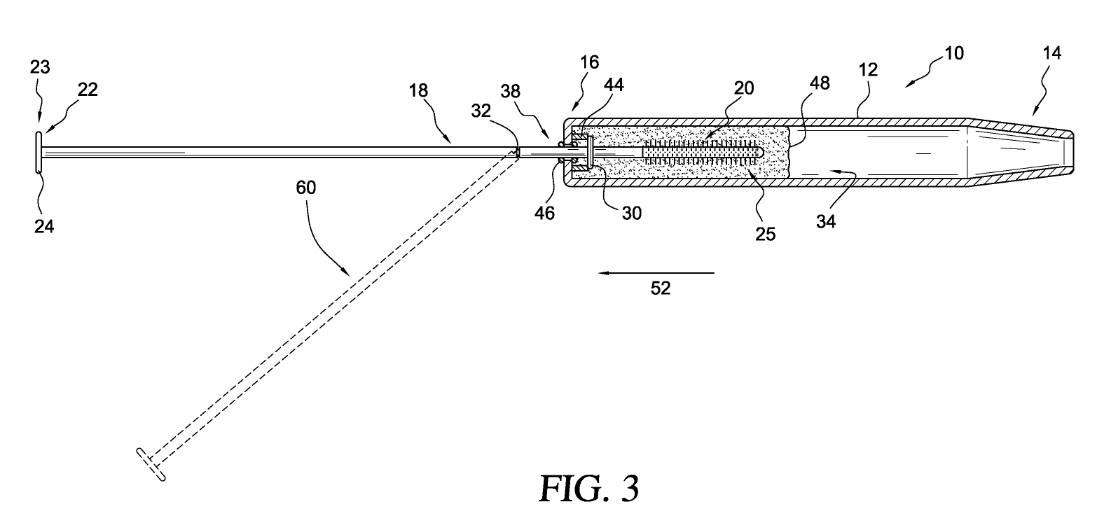

The weakened section 32 of the probe 18 may comprise a portion of the probe 18 that has been etched, scored, perforated, partially severed, and/or otherwise removed. The weakened section 32 may facilitate easy removal of the handle 22 once target cells have been obtained by the collector 20 and the probe 18 has been disposed in the storage position illustrated in FIG. 3. Alternatively, the probe 18 may be formed such that less probe material 18 is disposed at the weakened section 32. For example, the probe 18 may be stretched, bent, molded, and/or otherwise formed such that the probe 18 is thinner and, thus, weaker at the weakened section 32 than at other points along the length of the probe 18. Although the weakened section 32 may facilitate removal of the handle 22, the weakened section 32 may be formed so as not to diminish, for example, the efficacy of the medical device 10 and/or the functionality of any of the components discussed herein.

The handle 22 may be any shape, size, and/or other configuration useful in facilitating movement of the probe 18 relative to the sheath 12. For example, the handle 22 may be actuated in the direction of any of the arrows 50, 52, 54, 56 to facilitate corresponding movement of the probe 18 and the collector 20. The handle 22 may include one or more grips 24, sized, shaped, disposed, and/or otherwise configured to assist in manipulating the probe 18. The grip 24 may comprise, for example, a knob, lever, bump, ridge, lip, or other like structure to assist the user in digital manipulation of the probe 18. In an additional exemplary embodiment, the weakened section 32 of the probe 18 may comprise one or more release mechanisms such as, for example, a telescoping mechanism, a gripping device, corresponding threads, and/or other known mechanisms. In such an exemplary embodiment, the grip 24 may also comprise corresponding release mechanisms configured to assist in removing, breaking away, and/or otherwise releasing the handle 22 from the remainder of the probe 18. Such release mechanisms may include, for example, a spring-loaded push button release mechanism or other known devices.

The preservative 48 may be disposed within the lumen 34 to maintain integrity of the target cells removed by the collector 20 during the interim period between, for example, removal of the cells and subsequent analysis thereof. The preservative 48 may be any type of known saline, balanced salt, or other cell preservative. Such preservatives include, but are not limited to, Hanks' Balanced Salt solution, minimal essential tissue culture mediums, Polysal®, and Streck's Leuko Preserve products. The preservative 48 may be in substantially solid and/or substantially liquid form, and may resemble a gel-like substance suitable for preserving cells and/or tissue within a substantially closed and/or sealed environment such as within the lumen 34 of the sheath 12. The preservative 48 may substantially fill at least a portion or a length of the lumen 34. For example, the preservative 48 may substantially fill an inner diameter A of the lumen 34 around the probe 18. Any desired volume of the preservative 48 may be disposed within the lumen 34 and, in an exemplary embodiment, the volume of preservative 48 present may be sufficient to surround, envelop, and/or otherwise immerse substantially the entire collector 20, including the agitator 28 and the plurality of bristles 26 when the probe 18 and/or the collector 20 is disposed in the storage position illustrated in FIG. 3. The substantially fluid-tight seal between, for example, the sheath 12 and the probe 18 may assist in retaining the preservative 48 within the lumen 34 even when the probe 18 is moved repeatedly in the direction of arrows 50, 52, 54, 56. It is understood that the sheath 12 may further include additional sealing mechanisms such as, for example, the gaskets 46 or other mechanisms discussed above, to prohibit the preservative 48 from exiting the lumen 34 through, for example, the distal opening 36. Such sealing mechanisms may be disposed proximate the distal opening 36 or, alternatively, such mechanisms may be desirably located within the lumen 34 between the distal end 14 and the proximal end 16 of the sheath 12. Such additional sealing mechanisms may, for example, assist in retaining at least a portion and/or substantially all of the preservative 48 within a length of the lumen extending from the proximal end 16 of the sheath 12. Alternatively, as discussed above, the preservative 48 may be substantially gel-like in nature, and may be configured to substantially adhere to the walls of the lumen 34 so as to remain substantially proximate the proximal end 16 without adding additional structures to the sheath 12 for retaining the preservative 48 at a desirable location therein.

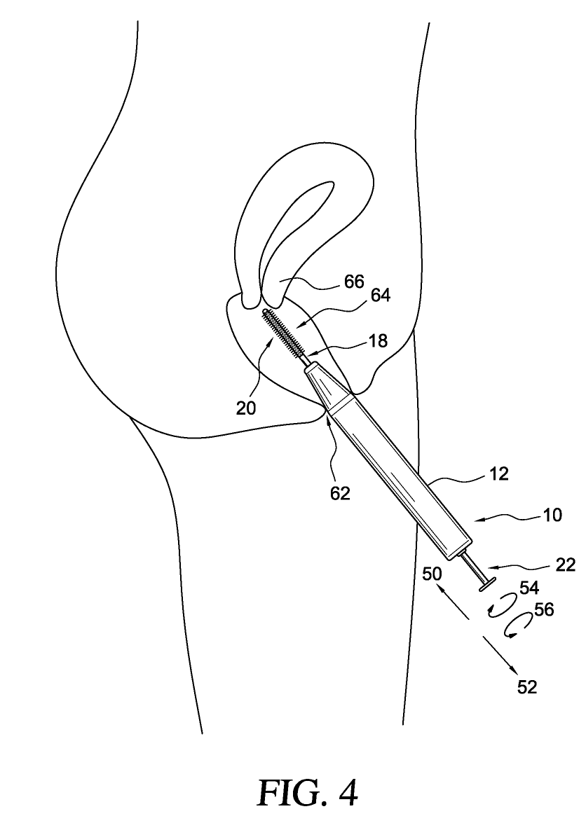

As shown in FIG. 4, the medical device 10 of the present disclosure may be used to guide insertion of the probe 18 into an anatomical opening or orifice 62 and/or for otherwise collecting one or more target cells from an anatomical structure 66. For example, as discussed above, the medical device 10 can be inserted into a female vagina for purposes of conducting a pap smear test, sexual transmitted disease test, or any other medical test or evaluation known in the art, and the uses of the present disclosure should not be limited to only such medical tests or evaluations. Accordingly, FIG. 4 merely illustrates a method of inserting the medical device 10 into an orifice 62 of the human body and is not intended to be an exact diagram of, for example, a female anatomy. FIG. 4 may differ slightly from an actual human anatomy.

While in the retracted position illustrated in FIG. 1, the medical device 10 may be at least partially inserted into a body cavity or canal 64 via the orifice 62. The orifice 62 may be, for example, a female vaginal opening. After the medical device 10 has been at least partially inserted into the canal 64, the handle 22 may be manipulated to extend the collector 22 through the distal opening 36 of the sheath 12 in the direction of arrow 50, and/or otherwise to transition the medical device 10 from the retracted position of FIG. 1 to the extended position illustrated in FIG. 2. In an exemplary embodiment, the user may advance the probe 18 in the direction of arrow 50 until the stop 30 contacts and/or abuts an inner wall 58 of the lumen 34. Alternatively, the probe 18 may be moved in the direction of arrow 50 until the collector 20 meets resistance from and/or contacts the anatomical structure 66 containing the desired target cells. In an exemplary embodiment, the anatomical structure 66 may be the female cervix and the desired target cells may be cervical cells. In such an exemplary embodiment, the collector 20 may be extended until it meets the opening of the cervix.

The handle 22 may then be rotated in the direction of arrows 54, 56 to encourage the collection of one or more target cells by the agitator 28 and/or the bristles 26 of the collector 20. It is understood that FIG. 4 is not drawn to scale and that FIG. 4 simply illustrates an exemplary method of inserting the medical device 10 into a body canal 64 and/or a method of collecting one or more target cells from anatomical structure 66. FIG. 4 is not intended to illustrate any specific anatomical or medical device dimensions, or insertion depths.

Once the target cells have been collected, and/or removed by the collector 20, the handle 22 may be moved in the direction of arrow 52, thereby transitioning the medical device from the extended position to the storage position illustrated in FIG. 3. By moving the handle 22 in the direction of arrow 52, the one or more target cells retained by the collector 20 may be disposed within the preservative 48. As shown in FIG. 3, the handle 22 may be moved in the direction of arrow 52 until the stop 30 contacts and/or abuts a portion of the sheath 12 such as, for example, one or more of the bumpers 44. When retracted in this way, the collector 20 may be substantially covered by, surrounded by, and/or otherwise immersed in the preservative 48.

Once in the storage position, the medical device 10 may be removed from the canal 64 via the orifice 62. The handle 22 may then be safely removed from the probe 18 by, for example, unscrewing, releasing, breaking-away, and/or otherwise detaching the handle 22 from the probe 18. In an exemplary embodiment, the handle 22 may be broken away from the remainder of the probe 18 by bending, snapping, breaking, and/or otherwise separating the handle 22 from the remainder of the probe 18 at the weakened section 32. The handle 22 may be removed by hand or, alternatively, additional implements such as, for example, scissors, or a knife blade may be utilized to facilitate the removal of the handle 22 from the probe 18. At least a portion of the medical device 10, such as, for example, the distal opening 36 of the sheath 12 may then be sealed with shrink wrap, a cap, an adhesive film, and/or any other known sealing structure to avoid contamination of the target cells disposed on the collector 20. The entire medical device 10 may then be placed in a container and sent to a remote laboratory or physician's office for analysis.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. A medical device, comprising:
(a) a sheath defining at least one lumen;
(b) a probe movably disposed within the lumen, the probe comprising a removable handle at a proximal end thereof, a collector at a distal end thereof, and a stop disposed between the proximal end and the distal end; and
(c) a preservative disposed within the lumen.

2. The medical device of claim 1, wherein the collector is a brush comprising a plurality of bristles.

3. The medical device of claim 1, wherein the collector comprises an agitator at the distal end configured to remove at least one target cell from an anatomical structure.

4. The medical device of claim 4, wherein the agitator comprises one of a knot, a loop, and a cup.

5. The medical device of claim 1, wherein the sheath forms a substantially fluid-tight seal with the probe at a proximal end of the sheath.

6. The medical device of claim 1, wherein the probe defines a weakened section proximal to the stop.

7. The medical device of claim 1, the probe having an extended position in which the collector is disposed outside of the lumen, a retracted position in which the collector is disposed within the lumen distal to the preservative, and a storage position in which the collector is disposed within the preservative.

8. The medical device of claim 7, wherein the lumen further includes a bumper, the bumper mating with the stop when the probe is in the storage position.

9. The medical device of claim 1, wherein a distal portion of the sheath has a smaller outer diameter than an outer diameter of a length of the sheath.

10. A medical device, comprising:
(a) a sheath defining a lumen;
(b) a probe disposed within the lumen, the probe having a collector at a distal end thereof, a break-away handle at a proximal end thereof, and a stop disposed between the collector and the handle; and
(c) a preservative substantially filling an inner diameter of the lumen around the probe.

11. The medical device of claim 10, wherein the preservative is substantially fluidly sealed within a proximal the lumen.

12. The medical device of claim 10, wherein the lumen forms a substantially fluid-tight seal with the probe at a distal end of the lumen.

13. The medical device of claim 10, wherein the probe defines a weakened section proximal to the stop.

14. The medical device of claim 10, the probe having an extended position in which the collector is disposed outside of the lumen, a retracted position in which the collector is disposed within the lumen distal to the preservative, and a storage position in which the collector is disposed within the preservative.

15. The medical device of claim 14, wherein the lumen further includes a bumper, the bumper mating with the stop when the probe is in the storage position.

16. A method of collecting a target cell from an anatomical structure, comprising:
(a) inserting a medical device into a body canal, the medical device comprising
a sheath defining at least one lumen,
a probe movably disposed within the lumen, the probe comprising a
removable handle at a proximal end thereof, a collector at a distal end thereof, and a stop disposed between the proximal end and the distal end, and
a preservative disposed within the lumen;
(b) collecting the target cell with the collector;
(c) disposing the target cell within the preservative; and
(d) removing the handle from the probe.

17. The method of claim 16, wherein collecting the target cell comprises extending the collector from the lumen.

18. The method of claim 16, wherein disposing the target cell within the preservative comprises moving the probe proximally, relative to the sheath, until the collector is substantially completely disposed within the preservative.

19. The method of claim 16, wherein collecting the target cell comprises rotating the probe relative to the sheath.

20. The method of claim 16, wherein the target cell is a cervical cell.
